(19) United States
(12) Patent Application Publication
(10) Pub. No.: US 2003/0045803 A1
(43) Pub. Date: Mar. 6, 2003

(54) Dynamic cardiac phantom and method of simulating cardiac motion

(76) Inventor: Kishore C. Acharya, Brookfield, WI (US)

Correspondence Address: ZIOLKOWSKI PATENT SOLUTIONS GROUP, LLC (GEMS), 14135 NORTH CEDARBURG ROAD, MEQUON, WI, 53097, US

(21) Appl. No.: 09/682,430

(22) Filed: Aug. 31, 2001

(51) Int. Cl. A61B 5/05

(52) U.S. Cl. 600/508

(57) ABSTRACT

An apparatus and method for simulating cardiac motion for use with an imaging system are provided. The apparatus includes an expandable balloon-like member having a number of protrusions fluidly connected thereto. Fluid is circulated between the pump and a fluid reservoir by a pump which is controlled by a programmable controller. Imaging data is acquired during circulation of the fluid between the reservoir and the phantom and subsequently analyzed to determine the effectacy and efficiency of the imaging protocol.

### BACKGROUND OF INVENTION

The present invention relates generally to simulating anatomical conditions of a patient and, more particularly, to an apparatus and method for simulating cardiac motion.

To properly develop and test imaging protocols and techniques it is necessary for researchers, software design engineers, and technicians to accurately and precisely simulate imaging conditions including general anatomical conditions of a typical patient, commonly referred to as “phantomming”. Known phantomming techniques include the use of animals to simulate the anatomical conditions of a human patient. Using animals to create a sample test condition has a number of disadvantages.

First, acquisition and care of animals often requires extensive and continual expenditures. Animals must be properly cared for and fed to maintain the health of the animal but also to be compliant with a number of regulations associated with animal testing. Further, in the absence of a well-trained animal, it is often necessary to sedate the animal, otherwise the animal may be uncooperative in simulating the test conditions. Moreover, the social climate associated with using animals and testing laboratories may be such that a company or research group may be publicly adversely affected by the use of animals in medical testing.

Additionally, researchers and design engineers forfeit a certain amount of control over the testing conditions by using animals to simulate human anatomical conditions. For example, without using expensive and potentially detrimental drugs to simulate precise human anatomical patterns, researchers and software designers are unable to exactly define and control the anatomical conditions, such as, defining cardiac motion. That is, researchers cannot reasonably force data to precisely mimic phase data taken from an echocardiograph (EKG) exam. Further, it is considerably more difficult for researchers and software engineers to repeat particular conditions using live animals.

Therefore, it would be desirable to design an apparatus and method for simulating cardiac motion that is repeatable, controllable, and cost efficient.

### SUMMARY OF INVENTION

A method and apparatus for simulating cardiac motion overcoming the aforementioned drawbacks are provided.

In accordance with one aspect of the present invention, a dynamic cardiac phantom is provided and includes a hollow chamber made of a pliable material to expand and contract based on an injection and discharge of fluid therein. The dynamic cardiac phantom further includes at least one inlet connected to the hollow chamber at one end and having another end connectable to a fluid source to inject and discharge fluid into the hollow chamber in a manner to simulate cardiac motion.

In accordance with another aspect of the present invention, a cardiac motion simulator for use with an imaging system is provided and includes a balloon having an inlet and a plurality of outlets. The inlet is configured to at least receive fluid. A plurality of tubes corresponding in number to the plurality of outlets of the balloon is also provided. Each tube includes an inlet connected to an outlet of the balloon wherein the plurality of tubes is further configured to receive fluid exiting the plurality of balloon outlets. Further, the balloon expands upon receipt of fluid and retracts upon discharge of fluid to mimic cardiac motion.

In accordance with another aspect of the present invention, a computer program for mimicking cardiac motion has instructions to supply fluid to an expandable chamber having a number of expandable tubes connected thereto. The computer program further includes instructions to slowly empty fluid from the expandable chamber and rapidly empty fluid from the expandable chamber. The computer program has further instructions to rapidly supply fluid to the expandable chamber and slowly supply fluid to the expandable chamber.

In accordance with a further aspect of the present invention, a method of phantomming cardiac motion for use with a scanner is provided. The method includes the step of connecting a balloon having an inlet and a number of tubular protrusions to a fluid reservoir. The method further includes the steps of filling the balloon with fluid and circulating fluid to and fro the balloon. Imaging data is then acquired of the balloon during circulation of the fluid to and from.

In accordance with yet a further aspect of the present invention, a computer tomography system having a rotatable gantry having an opening is provided. The CT system further includes a high frequency electromagnetic energy projection source configured to project high frequency energy toward an object and a scintillator array having a plurality of scintillators to receive high frequency electromagnetic energy attenuated by the object. A photodiode ray is also provided and includes a plurality of photodiodes wherein the photodiode ray is coupled to the scintillator ray and configured to detect light energy emitted there from. A plurality of electrical interconnects are configured to transmit photodiode outputs to a data processing system and a computer is also provided to produce a visual display based upon the photodiode outputs transmitted to the data processing system. The object is defined to include an expandable balloon having a number of tubular protrusions and an inlet configured to receive circulating fluid such that circulation of the fluid simulates cardiac motion.

Various other features, objects and advantages of the present invention will be made apparent from the following detailed description and the drawings.

### BRIEF DESCRIPTION OF DRAWINGS

The drawings illustrate one preferred embodiment presently contemplated for carrying out the invention.

In the drawings:

FIG. 1 is a pictorial view of a CT imaging system;

FIG. 2 is a block diagram of the system illustrated in FIG. 1;

FIG. 3 is a schematic representation of a dynamic cardiac phantom in accordance with the present invention.

FIG. 4 is a flow chart for simulating dynamic cardiac motion in accordance with the present invention.

### DETAILED DESCRIPTION

The operating environment of the present invention is described with respect to a four-slice computed tomography (CT) system. However, it will be appreciated by those of ordinary skill in the art that the present invention is equally applicable for use with single-slice or other multi-slice configurations. Moreover, the present invention will be described with respect to the detection and conversion of x-rays. However, one of ordinary skill in the art will further appreciate, that the present invention is equally applicable for use with other imaging modalities, such as Magnetic Resonance Imaging (MRI), Positron Emission Tomography (PET), or x-ray.

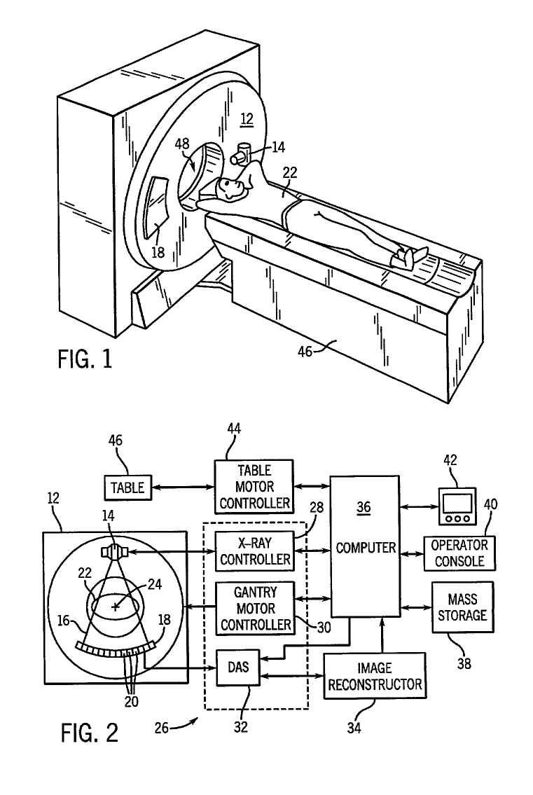

Referring to FIGS. 1 and 2, a computed tomography (CT) imaging system 10 is shown as including a gantry 12 representative of a “third generation” CT scanner. Gantry 12 has an x-ray source 14 that projects a beam of x-rays 16 toward a detector array 18 on the opposite side of the gantry 12. Detector array 18 is formed by a plurality of detectors 20 which together sense the projected x-rays that pass through a medical patient 22. Each detector 20 produces an electrical signal that represents the intensity of an impinging x-ray beam and hence the attenuated beam as it passes through the patient 22. During a scan to acquire x-ray projection data, gantry 12 and the components mounted thereon rotate about a center of rotation 24.

Rotation of gantry 12 and the operation of x-ray source 14 are governed by a control mechanism 26 of CT system 10. Control mechanism 26 includes an x-ray controller 28 that provides power and timing signals to an x-ray source 14 and a gantry motor controller 30 that controls the rotational speed and position of gantry 12. A data acquisition system (DAS) 32 in control mechanism 26 samples analog data from detectors 20 and converts the data to digital signals for subsequent processing. An image reconstructor 34 receives sampled and digitized x-ray data from DAS 32 and performs high speed reconstruction. The reconstructed image is applied as an input to a computer 36 which stores the image in a mass storage device 38.

Computer 36 also receives commands and scanning parameters from an operator via console 40 that has a keyboard. An associated cathode ray tube display 42 allows the operator to observe the reconstructed image and other data from computer 36. The operator supplied commands and parameters are used by computer 36 to provide control signals and information to DAS 32, x-ray controller 28 and gantry motor controller 30. In addition, computer 36 operates a table motor controller 44 which controls a motorized table 46 to position patient 22 and gantry 12. Particularly, table 46 moves portions of patient 22 through a gantry opening 48.

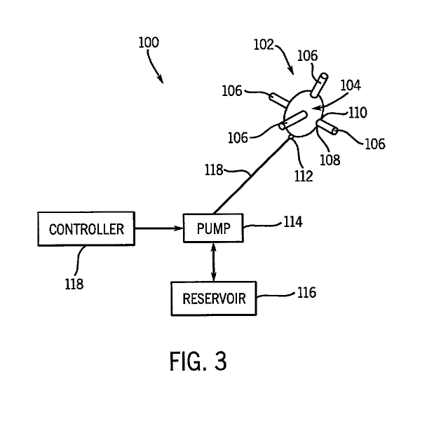

The present invention provides an apparatus and method of simulating cardiac motion for use with an imaging scanner, such as a CT system. Referring to FIG. 3, the cardiac motion simulator 100 includes a phantom 102. Phantom 102 is formed of an expandable material and includes a hollowed center region 104 and a number of tubular protrusions 106. In a preferred embodiment, there are four tubular protrusions 106 corresponding to the four chambers of a human heart. Protrusions 106 may be fastenly connected to an outer surface of the center region 104 or seamlessly connected, i.e., the protrusions 106 and the center region 104 formed as a single unitized structure. However, the inlets 108 of the tubular protrusions must be in fluid communication with the outlets 110 of the center region 104. Phantom 102 further includes a fluid inlet 112 that fluidly communicates with center region 104.

Simulator 100 further includes a pump 114 configured to supply fluid to the phantom 102 from a fluid reservoir 116 via supply/discharge pipe 118. To mimic cardiac motion, the pump cyclically circulates fluid between the phantom 102 and the reservoir 116. In a preferred embodiment, the cyclical circulation of fluid between phantom 102 and reservoir 116 are such that the volume changing phases of the heart are simulated. That is, the pump circulates the fluid to mimic a slow empty phase, a rapid empty phase, a change-over to filling phase or pause phase, a rapid filling phase, a slow filling phase, and a change-over to empty phase or pause phase. Typically, the rapid filling phase fills the phantom with fluid at a speed twice that of the slow filling phase. Conversely, the rapid empty phase discharges fluid from the phantom at a speed 50% slower than the fast filling phase. These values are simply one preferred embodiment and are used for illustrative purposes only and are not intended to limit the scope nor the breadth of the present invention.

Simulator 100 further includes a controller 118 configured to transmit modulating signals to the pump thereby instructing the pump to circulate fluid between phantom 102 and reservoir 116. The controller 118 may include a computer program that automatically causes the pump to circulate fluid to the phantom and reservoir, but also may transmit operator entered parameters to the pump 114. For example, controller 118 may include a computer program that automatically randomly varies the aforementioned phases to more accurately reflect cardiac motion. Additionally, a user such as a researcher or design engineer may wish to simulate varied cardiac motion and therefore may input cardiac motion data directly to the controller using a keypad and/or keyboard. Other known data input techniques and modules are contemplated and are within the scope of the present application.

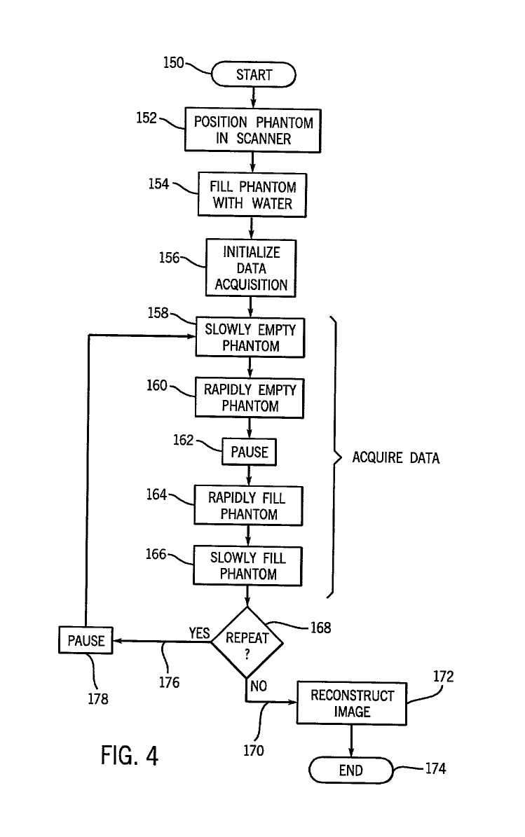

Accordingly, the present invention includes an algorithm for simulating cardiac motion for use with an imaging system. The present invention contemplates both a method of simulating cardiac motion and a computer program implementing the steps of the method, and both will be discussed with reference to FIG. 4. The algorithm begins at 150 with positioning of the phantom within a field-of-view of the scanner 152. Once positioned in the scanner 152, the phantom is filled with fluid such as water at 154. A data acquisition sequence is then initialized at 156 followed by a slow emptying of the water from the phantom at 158. In a preferred embodiment the slow emptying phase 158 lasts for 100 ms. per 1,000 ms. of total phase time. Following the slow emptying phase 158, water is rapidly drained fromm the phantom at 160 for a period of 150 ms. per 1,000 ms. After rapidly emptying the phantom at 160, the algorithm pauses at 162 for a period of 50 ms. per 1,000 ms. as the algorithm changes over fromm the emptying phase to the filling phase. Thereafter at 164, the phantom is rapidly filled with water for a period of 100 ms. per 1,000 ms. followed at 166 by a slow filling phase lasting 500 ms. per 1,000 ms. At 168, the algorithm determines if simulation of cardiac motion is to continue. That is, if simulation is complete 168, 170, an image is reconstructed of the phantom at 172 in accordance with known imaging techniques whereupon the algorithm ends at 174. However, if cardiac motion simulation is not complete and is to be repeated 168, 176, the algorithm pauses at 178 for a period of 100 ms. per 1,000 ms. to accommodate changing over to the emptying phase fromm the filling phase. Following pausing 178, the algorithm begins anew at 158 with the slowly emptying of water from the phantom. It should be noted that imaging data is continuously acquired during emptying/filling phases 158-166.

The time period associated with each phase 158-166 is for illustrative purposes only and is not intended to limit the scope or breadth of the instant application. Moreover, the present invention contemplates varying of circulation phases 158-166 fromm cycle to cycle, and such variation may be as much as 20% in a preferred embodiment to more precisely simulate cardiac motion of a patient's heart. The amount of variation however is preferably random and based upon a uniform distribution. Additionally, the algorithm of FIG. 4 contemplates using analyzed EKG traces from a real patient to determine the phase times for acts 158-166. By using actual EKG data, the algorithm may more accurately reflect actual cardiac motion thereby providing more reliable and resourceful data for image reconstruction and subsequent software development.

That is, the final reconstructed image of the cardiac phantom is analyzed to determine what changes, if any, should be made to the imaging protocol so that artifact free images result. The present invention contemplates evaluation of the imaging protocols using the cardiac phantom during the protocol development stage as well as during onsite clinical trials of the imaging protocol.

In accordance with one embodiment of the present invention, a dynamic cardiac phantom is provided and includes a hollow chamber made of a pliable material to expand and contract based on an injection and discharge of fluid therein. The dynamic cardiac phantom further includes at least one inlet connected to the hollow chamber at one end and having another end connectable to a fluid source to inject and discharge fluid into the hollow chamber in a manner to simulate cardiac motion.

In accordance with another embodiment of the present invention, a cardiac motion simulator for use with an imaging system is provided and includes a balloon having an inlet and a plurality of outlets. The inlet is configured to at least receive fluid and preferably discharge fluid as well. A plurality of tubes corresponding in number to the plurality of outlets of the balloon is also provided. Each tube includes an inlet connected to an outlet of the balloon wherein the plurality of tubes is further configured to receive fluid exiting the plurality of balloon outlets. Further, the balloon is configured to expend upon receipt of fluid and retract upon discharge of fluid to mimic cardiac motion.

In accordance with yet another embodiment of the present invention, a computer program for mimicking cardiac motion has instructions to supply fluid to an expandable chamber having a number of expandable tubes connected thereto. The computer program further includes instructions to slowly empty fluid from the expandable chamber and rapidly empty fluid from the expandable chamber. The computer program has further instructions to rapidly supply fluid to the expandable chamber and slowly supply fluid to the expandable chamber.

In a further embodiment of the present invention, a method of phantomming cardiac motion for use with a scanner is provided. The method includes the step of connecting a balloon having an inlet and a number of tubular protrusions to a fluid reservoir. The method further includes the steps of filling the balloon with fluid and circulating fluid to and fro the balloon. Imaging data is then acquired of the balloon during circulation of the fluid to and from.

In yet a further embodiment of the present invention, a computer tomography system having a rotatable gantry having an opening is provided. The CT system further includes a high frequency electromagnetic energy projection source configured to project high frequency energy toward an object and a scintillator ray having a plurality of scintillators to receive high frequency electromagnetic energy attenuated by the object. A photodiode ray is also provided and includes a plurality of photodiodes wherein the photodiode ray is coupled to the scintillator ray and configured to detect light energy emitted therefrom. A plurality of electrical interconnects are configured to transmit photodiode outputs to a data processing system and a computer is also provided to produce a visual display based upon the photodiode outputs transmitted to the data processing system. The object is defined to include an expandable balloon having a number of tubular protrusions and an inlet configured to receive circulating fluid such that circulation of the fluid simulates cardiac motion.

The present invention has been described in terms of the preferred embodiment, and it is recognized that equivalents, alternatives, and modifications, aside from those expressly stated, are possible and within the scope of the appending claims.

What is claimed is:

1. A dynamic cardiac phantom comprising:
a hollow chamber made of pliable material to expand and contract based on an injection and discharge of fluid therein; and
at least one inlet connected to the hollow chamber at one end and having another end connectable to a fluid source to inject and discharge fluid into the hollow chamber in a manner to simulate cardiac motion.

2. The dynamic cardiac phantom of claim 1 wherein the hollow chamber has a shape to simulate that of a heart.

3. The dynamic cardiac phantom of claim 2 wherein the hollow chamber has a plurality of protrusions extending therefrom, the plurality of protrusions having a shape to simulate a plurality of chambers of the heart.

4. The dynamic cardiac phantom of claim 1 wherein the fluid includes water.

5. The dynamic cardiac phantom of claim 1 wherein the at least one inlet is further configured to inject and discharge fluid into the hollow chamber in a plurality of modes, the plurality of modes including a slow drain, a rapid drain, a change-over to supply, a rapid supply, a slow supply, and a change-over to drain.

6. The dynamic cardiac phantom of claim 5 wherein the slow drain mode is 100 per 1,000 ms., the rapid drain mode is 150 ms. per 1,000 ms., the change-over to supply mode is 50 ms. per 1,000 ms., the rapid supply mode is 100 ms. per 1,000 ms., the slow supply mode is 500 ms. per 1,000 ms., and the change-over to drain mode is 100 ms. per 1,000 ms.

7. A cardiac motion simulator for use with an imaging system, the simulator comprising:
a balloon having an inlet and a plurality of outlets, the inlet configured to at least receive fluid; and
a plurality of tubes corresponding in number to the plurality of outlets, each tube having an inlet connected to an outlet of the balloon, wherein the plurality of tubes is configured to receive fluid exiting the plurality of balloon outlets;
wherein the balloon expands upon receipt of fluid and retracts upon discharge of fluid to mimic cardiac motion.

8. The cardiac motion simulator of claim 7 further comprising a pump connected to the balloon inlet and configured to supply fluid and drain fluid from the balloon and plurality of tubes.

9. The cardiac motion simulator of claim 8 wherein the pump is configured to operate in a plurality of modes that includes a cyclically slow drain, a rapid drain, change-over to supply, a rapid supply, a slow supply, and a change-over to drain the balloon and the plurality of tubes in mimicking cardiac motion.

10. The cardiac motion simulator of claim 9 wherein the pump is further configured to slowly drain the balloon and the plurality of tubes for 100 ms. per 1,000 ms., rapidly drain the balloon and the plurality of tubes for 150 ms., per 1,000 ms., change-over to supplying for 50 ms. per 1,000 ms., rapidly supply fluid to the balloon and the plurality of tubes for 100 ms. per 1,000 ms., slowly provide fluid to the balloon and the plurality of tubes for 500 ms. per 1,000 ms., and change-over to draining for 100 ms. per 1,000 ms.

11. The cardiac motion simulator of claim 10 wherein the pump is further configured to vary the supplying, change-over, and draining for each cycle.

12. The cardiac motion simulator of claim 8 further comprising a supply pipe configured to transport fluid between the balloon inlet and the pump.

13. The cardiac motion simulator of claim 8 wherein the fluid is water and further comprises a control configured to regulate the supplying and draining of fluid by the pump.

14. The cardiac motion simulator of claim 13 wherein the control is further configured to regulate the supplying and draining of fluid by the pump using EKG traces of a patient.

15. The cardiac motion simulator of claim 14 wherein the control has a programmable storage medium and is configured to modulate the pump using stored EKG data.

16. A computer readable storage medium having thereon a computer program representing a set of instructions that when executed by a computer causes the computer to:
(A) supply fluid to an expandable chamber having a number of expandable tubes connected thereto;
(B) slowly empty fluid from the expandable chamber;
(C) rapidly empty fluid from the expandable chamber;
(D) rapidly supply fluid to the expandable chamber; and
(E) slowing supply fluid to the expandable chamber.

17. The computer readable storage medium of claim 16 wherein the set of instructions further causes the computer to cyclically repeat any act (B) through (E) so as to simulate heart motion in a patient.

18. The computer readable storage medium of claim 17 wherein the set of instructions further causes the computer to empty fluid in acts (B) and (C) and supply fluid in acts (D) and (E) based on EKG traces from a patient.

19. The computer readable storage medium of claim 17 wherein the set of instructions further causes the computer to determine a total cycle time period and a time period for each of:
slowly emptying fluid;
rapidly emptying fluid;
rapidly supplying fluid; and
slowly supplying fluid.

20. The computer program of claim 19 wherein the time period for slowly emptying fluid is 10% of the total cycle time period, the time period for rapidly emptying fluid is 15% of the total cycle time period, the time period for rapidly supplying fluid is 10% of the total cycle time period, and the time period for slowly supplying fluid is 50% of the total cycle time period.

21. The computer readable storage medium of claim 19 wherein the set of instructions further causes the computer to determine a change-over time period between the rapidly emptying fluid time period and the rapidly supplying fluid time period and another change-over time period between the slowly supplying fluid time period and the slowly emptying fluid time period.

22. The computer readable storage medium of claim 21 wherein the change-over time period is 5% of the total cycle time period and the another change-over time period is 10% of the total cycle time period.

23. The computer readable storage medium of claim 22 wherein the set of instructions further causes the computer to vary each time period with each repetition of acts (B) through (E).

24. The computer readable storage medium of claim 23 wherein the set of instructions further causes the computer to vary each time period with each repetition of acts (B) through (E) by 20%.

25. A method of phantomming cardiac motion for use with a scanner, the method comprising:
connecting a balloon having an inlet and a number of tubular protrusions to a fluid reservoir;
filling the balloon with fluid;
circulating fluid to and from the balloon; and
acquiring imaging data of the balloon during the circulation step.

26. The method of claim 25 further comprising generating a reconstructed image from the acquired imaging data and determining artifact presence in the reconstructed image.

27. The method of claim 26 further comprising repeating the circulating, acquiring, and generating steps using at least one of a new acquisition protocol and a new image reconstruction protocol designed to reconstruct an image with reduced artifacts.

28. The method of claim 25 further comprising circulating the fluid in accordance with a number of defined phases.

29. The method of claim 28 wherein the number of defined phases include:
a slow empty phase;
a rapid empty phase;
change-over to filling phase;
rapid filling phase;
slow filling phase; and
charge over to empty phase.

30. The method of claim 29 further comprising defining the number of defined phases using analyzed data from an EKG of a patient.

31. The method of claim 25 wherein the circulating further comprises:
slowly emptying fluid from the expandable chamber for 100 ms. per 1,000 ms.;
rapidly emptying fluid from the expandable chamber for 50 ms. per 1,000 ms.;
pausing for 50 ms per 1,000 ms.;
rapidly filling the expandable chamber for 100 ms. per 1,000 ms.; slowly filling the expandable chamber for 500 ms. per 1,000 ms.; and pausing again for 100 ms. per 1,000 ms.

32. The method of claim 25 further comprising programming a computer to regulate the circulating of fluid to and from the balloon.

33. The method of claim 25 wherein the scanner includes at least one of a CT scanner, an NM/PET scanner, an MRI scanner, and an x-ray scanner.

34. A computed tomography system comprising:
a rotatable gantry having an opening;
a high frequency electromagnetic energy projection source to project high frequency energy toward an object;
a scintillator array having a plurality of scintillators to receive high frequency electromagnetic energy attenuated by the object;
a photodiode array having a plurality of photodiodes, wherein the photodiode array is optically coupled to the scintillator array and is configured to detect light energy emitted therefrom;
a plurality of electrical interconnects configured to transmit photodiode outputs to a data processing system;
a computer to produce a visual display based upon the photodiode outputs transmitted to the data processing system; and
wherein the object includes an expandable balloon having a number of tubular protrusions and an inlet configured to receive circulating fluid such that circulation of the fluid simulates cardiac motion.

35. The computed tomography system of claim 34 further comprising a pump and a fluid reservoir, the pump configured to cyclically provide and drain fluid from the balloon.

36. The computed tomography system of claim 34 further comprising a programmable control configured to modulate the pump to provide cardiac motion simulation.
